(19) United States
(12) Patent Application Publication
(10) Pub. No.: US 2023/0140078 A1
(43) Pub. Date: May 4, 2023

(54) SPIRULINA DIGESTIBLE AND BIOAVAILABLE PROTEIN MILK COMPOSITION

(71) Applicant: WELLISEN INC., Reston, VA (US)

(72) Inventors: Jayant DESHPANDE, Ramsey, NJ (US); Benedict BRUNO, Mysore (IN); Yuvraj PURI, Great Falls, VA (US)

(21) Appl. No.: 17/514,506

(22) Filed: Oct. 29, 2021

(51) Int. Cl. A23C 11/10; A23C 1/04

(52) U.S. Cl. CPC A23C 11/10; A23C 1/04; A23C 2240/15

(57) ABSTRACT

An alternative milk composition of spirulina digestible, bioavailable proteins, vitamins and minerals is described. The proteins, vitamins and minerals present in spirulina biomass are rendered digestible and bioavailable by unique homogenization process followed by thermo-chemical treatment. The alternative milk made with digestible, bioavailable proteins, vitamins and minerals is formulated with colorant, sweetener, flavor and gelling agent. The milk so obtained is packed with digestible and bioavailable proteins, vitamins and minerals is free from fishy odor and bitter taste.

### FIELD OF INVENTION

Described herein is a formulation of spirulina enriched proteins that provides readily digestible and bioavailable proteins, vitamins and minerals as a source of complete nutrition in the form non-dairy milk. Particularly, described herein is an aqueous extract of homogenized spirulina biomass to serve as a source of readily digestible proteins. More particularly, described herein is an aqueous extract of spirulina which is rich in essential amino acids along with other nutrients such as vitamins, minerals, flavonoids, polyphenols etc. The aqueous extract of Spirulina described herein serves as a source of readily digestible proteins when formulated as ready-to-drink, non-dairy, vegan milk composition. A milk composition as described herein, which is rich in essential and non-essential amino acids along with vitamins and minerals, provides readily digestible source of these nutrients to all age groups.

### BACKGROUND

Spirulina represents a genus of cyanobacteria (blue-green algae) that can be consumed by humans and other animals. Two species of spirulina are Arthrospira platensis and A. maxima. The common name, spirulina, refers to the dried biomass of A. platensis, a photosynthetic bacteria that covers the groups Cyanobacteria and Prochlorophyta. Scientifically, a distinction exists between spirulina and the genus Arthrospira.

Arthrospira species are free-floating, filamentous cyanobacteria characterized by cylindrical, multicellular trichomes in an open left-handed helix. They occur naturally in tropical and subtropical lakes with high pH and high concentrations of carbonate and bicarbonate. Species of Arthrospira have been isolated from alkaline brackish and saline waters in tropical and subtropical regions. Among the various species included in the genus Arthrospira, A. platensis is the most widely distributed and is mainly found in Africa, Asia, and South America, whereas A. maxima is confined to Central America, Mexico, and California. Most cultivated spirulina is produced in open-channel raceway ponds, with paddle wheels used to agitate the water.

Provided in its typical supplement form as a dried powder, a 100-g amount of spirulina supplies 290 Caloriesand is a rich source (20% or more of the Daily Value, DV) of numerous essential nutrients, particularly protein, B vitamins (thiamin and riboflavin, 207% and 306% DV, respectively), and dietary minerals, such as iron (219% DV) and manganese (90% DV). The lipid content of spirulina is 8% by weight providing the fatty acids, gamma-linolenic acid, alpha-linolenic acid, linoleic acid, stearidonic acid,^{]}eicosapentaenoic acid (EPA), docosahexaenoic acid (DHA), and arachidonic acid. In contrast to those 2003 estimates (of DHA and EPA each at 2 to 3% of total fatty acids), 2015 research indicated that spirulina products “contained no detectable omega-3 fatty acids” (less than 0.1%, including DHA and EPA). An in vitro study reported that different strains of microalgae produced DHA and EPA in substantial amounts.

Allergy, Rhinitis, and Immunomodulation: It has been well documented that spirulina exhibits anti-inflammatory properties by inhibiting the release of histamine from mast cells.

In a recent randomized, double-blind placebo-controlled trial, individuals with allergic rhinitis were fed daily, either with placebo or spirulina for 12 weeks. Peripheral blood mononuclear cells were isolated before and after the spirulina feeding and levels of cytokines (interleukin-4 (IL-4), interferon-y (IFN-y) and interleukin-2), which are important in regulating immunoglobulin (lg)E-mediated allergy, were measured. The study showed that high dose of spirulina significantly reduced IL-4 levels by 32%, demonstrating the protective effects of this microalga toward allergic rhinitis. Ishii et al. studied the influence of Spirulina on IgA levels in human saliva and demonstrated that it enhances IgA production, suggesting a pivotal role of microalga in mucosal immunity.

A Japanese team identified the molecular mechanism of the human immune capacity of spirulina by analysing blood cells of volunteers with pre- and post-oral administration of hot water extract of Spirulina platensis. IFN-y production and Natural Killer (NK) cell damage were increased after administration of the microalga extracts to male volunteers.

In a recent double-blind, placebo-controlled study from Turkey evaluating the effectiveness and tolerability of spirulina for treating patients with allergic rhinitis, spirulina consumption significantly improved the symptoms and physical findings compared with placebo, including nasal discharge, sneezing, nasal congestion and itching.

It is well understood that deficiency of nutrients is responsible for changes in immunity, which manifests as changes in production of T-cells, secretory IgA antibody response, cytokines and NK-cell activity. The above studies suggest that Spirulina may modulate the immune system by its role in covering nutritional deficiencies.

### Extraction and Purification of Proteins From Spirulina

Several techniques for the isolation of proteins from spirulina biomass are known in the art. Protein solubility is pH dependent. Highly acidic and alkaline conditions enhance the solubility of algal proteins by inducing net charges on the amino acid residues. Proteins are least soluble at their isoelectric pH and precipitate out. Thus, solubilization under alkaline conditions followed by precipitation at isoelectric pH is a useful strategy for obtaining crude protein isolates. Several authors reported protein extraction from green algae and cyanobacteria using this method. Other parameters that could impact protein solubility include extraction (solubilization or precipitation) time, solvent/biomass ratio (biomass concentration), and temperature. High temperature causes protein denaturing and also increases the energy input required for the overall process. Hence, heat treatment is undesirable in protein isolation processes.

CN108753874A describes Preparation method of micromolecular active peptide novel spirulina powder. The invention discloses a preparation method of micromolecular active peptide novel spirulina powder. Impurities in spirulina can be decomposed by performing pre-freezing, drying, screening, smashing, fermenting for deodorization and wall breaking and combining with chemical enzyme reaction technology for enzymolysis, and the micromolecular active peptide novel spirulina powder after enzymolysis has the advantages of being easy for digestion and absorption, small in molecular weight, less prone to denaturing at high temperature and in acidic-alkaline conditions, high in instant solubility, free of fishy smell, good in taste, easy for dissolution and absorption, capable of maintaining all original nutritional ingredients and low in preparation cost, can be quickly absorbed in small intestines and is unaffected by digestion dysfunction. The novel spirulina powder has physiological functions like resisting radiation, promoting lipid metabolism, lowering cholesterol, lowering blood pressure, resisting fatigue, improving body immunity, promoting human metabolism, relieving hangover and resisting oxidation and quicker in treatment

CN108822188A Protein extraction and separation purification method. The invention discloses a protein extraction and separation purification method. The method includes the steps of: conducting freezing embrittlement on spirulina subjected to low temperature wall-breaking, and then performing ultrasonic wall-breaking treatment; adding a 0.1 mol/L NaCl solution and 711 anion exchange resin, conducting pulp grinding for 0.5 min, performing filtering, adjusting the pH value to 7.1-7.3, and conducting heating and cooling, then performing centrifugation, taking the supernatant, carrying out ammonium sulfate salting-out, taking 45%-55% of the salted out protein precipitate section to obtain precipitate A; dissolving the precipitate A with a column balanced buffer solution, then letting the obtained product pass through a sephadex G-50 column balanced by the buffer solution, conducting elution, collecting the 6th-7thml eluent, and conducting dialysis and freeze-drying, thus obtaining spirulina protein powder. Through the perfect combination of secondary wall-breaking, the dissolution rate of spirulina protein is 80.31%-81.21%, thus effectively improving the extraction rate of spirulina protein, and enhancing the purity of the obtained spirulina protein

CN109393489A Selenium-enriched spirulina powder and preparation method thereof The invention relates to selenium-enriched spirulina powder and a preparation method thereof. The selenium-enriched spirulina powder consists of the following components of 60-80 parts of a spirulina extract, 20-30 parts of buckwheat, 10-15 parts of a Chinese wolfberry fruit extract, 2-6 parts of flos sophorae rutin flavone and 6-10 parts of a selenium-enriched yeast extract, wherein an extraction method of the flos sophorae rutin flavone comprises the following steps of S1, crushing raw materials: drying flos sophorae rutin raw materials until the water ratio is lower than 8%, performing crushing, and performing screening with a 60-80-mesh sieve; S2, compounding a dual-aqueous phase extraction solution, wherein the dual-aqueous phase extraction solution consists of the following components in percentage by weight: 15-25% of hexamethyl phosphoramide, 5-8% of potassium carbonate, 3-6% of ammonium sulphate and the balance of water; S3, performing extraction: mixing raw material powder crushed in the step S1 with the extraction solution compounded in the step S2 at 40-60 DEG C, performing stirring for 0.5-1 h, performing standing for 30-45 min, and performing layering; and S4, performing separation: taking an upper layer organic phase, performing concentration, and separating out crystals so as to obtain the flos sophorae rutin flavone.

CN110810712A discloses a preparation method of a spirulina deodorant noodle product. The method for deodorizing spirulina in this noodle product is to add 2-3 parts of spirulina and 1 part of whole egg powder to 50-60 parts of water according to the massration, add Aspergillus oryzae neutral protease to hydrolyze for 30-60 minutes, and then kill the enzyme. The spirulina deodoranthydrolysate was obtained. Mix the spirulina deodorant hydrolysate with flour and auxiliary materials evenly, knead it into a smooth dough, and then ferment and steam it to make steamed bread; or ferment, shape, proof and bake the above dough to make bread. The invention can effectively remove the algae smell of spirulina, and the prepared steamed bread and bread have good taste and emerald green color.

International Journal of Innovative Technology and Exploring Engineering (IJITEE)ISSN: 2278-3075,Volume-8 Issue-12, October 2019 describes extraction of protein from green spirulina microalgae platensis was carried out by centrifugation, ultra sound assisted extraction and combination of centrifugation and ultrasound assisted extraction spirulina platensis was carried out using ultrasound-assisted extraction and statistical optimization method is used to obtain the optimum conditions. A Box-Behnkendesign method was used to optimize the process conditions affecting protein extraction. This condition investigated on microalgae cell disruption method by the combination of both ultrasonication and centrifuge. Different buffer solutions were used and the time of the ultrasonication was also varied. The protein extraction quantity was evaluated. The obtained results confirm that mixed buffer III showed the high concentration of protein but low quality. The study on the effect of ultrasonication period, the concentration of protein increased and remains constant also when duration of sonication is elongated and this result was observed during continuous ultrasonication in similar manner. The protein concentration is one of the important aspect, the protein quality must be satisfied. The results confirms the optimum condition of protein extraction from green microalgae requires the combination of the use of buffer solution and a proper duration of ultrasonication to maximize the protein

IN 201711038189 provides a physical process for preparation of soluble whole Spirulina powder comprising the steps of :i. Suspending the freshly harvested and washed wet biomass of Spirulina in potable water so as to obtainslurrywith 8 to 10% total solids (TS).ii. Subjecting the Spirulina slurry obtained in step (i) to homogenization up-to 800 bar pressure at maximum temperature of 20° C. and maximum flow rate of 250 mL min-1to disrupt the cell wall and facilitate the release of the intracellular components of Spirulina comprising of hydrophilic components including proteins, phycocyanin, carbohydrates, vitamins and minerals), lipids and lipophilic components including chlorophyll and carotenoids in aqueous phase to form whole Spirulina emulsion. iii. Subjecting the whole Spirulina emulsion obtained in step (ii) to homogenization up-to 800 bar pressure at temperature ranging from 5 to 20° C. for a minimum of 2 cycles at a flow rate of 150 to 250 mL min-1to achieve uniform dispersion of lipids and lipophilic intracellular components such as chlorophyll and carotenoids, of whole Spirulina emulsion nto protein matrix in the aqueous phase which also contains phycocyanin, carbohydrates, vitamins and minerals, thus obtaining a stable whole Spirulina emulsion (iv. Centrifugation of the stable whole Spirulina emulsion obtained in step (iii) up to 5,000 rpm for a period of 5to 10 minutes to remove cell wall debris and obtain a clarified stable whole Spirulina emulsion having 3 to 5% TS which is devoid of any gravimetric settling or phase separation. v. Drying of the clarified stable whole Spirulina emulsion obtained in step (iv) by freeze drying/spray drying to obtain the soluble whole Spirulina powder.

WO2016/015013 describes a method of producing a protein material comprising, exposing a delipidated biomass that contains a proto-protein to acidic conditions by adjusting the pH of the biomass to a depressed pH of less than 4.5 and holding the pH of the biomass at said depressed pH for atleast10 minutes to convert the proto-protein into the protein material

CN101366404A describes a method for preparing spirulina flour. The invention relates to a method for preparing spirulina flour. The method comprises the following steps: flour directly passes through a flat sieve to sieve out excellent flour to enter a packing auger; the packing auger stirs the flour uniformly; spirulina powder is filled into a feeder and the flat sieve; the sieved excellent flour simultaneously enters two packing augers to ensure that the flour and the spirulina powder are stirred more uniformly; the mixed flour is brought into a container by a hoisting machine, and is packaged into a finished product by an electronic quantitative packing scale. With the method, the spirulina powder and the flour are combined to be easily digested by a human body, so that physiological functions of the human body can be balanced to promote metabolism and improve immunity; moreover, the spirulina powder and the flour can be stirred more uniformly by the two packing augers. The spirulina flour is rich in comprehensive nutrition, such as protein, chlorophyll, vitamin, mineral, microelement and the like

CN1344514A describes Spirulina powder producing process Spirulina has protein content as high as 50-70 %, reasonable amino acid proportion and rather high contents of spirulina polysaccharide, vitamins and trace elements. The spirulina powder produced by using spray drying process of prior art results in high temperature damage of the activity and effective components. The present invention provides one vacuum freezing and sublimating drying process so as to maintain the activity and effective components of spirulina. The spirulina powder as one kind of health product has high nutrient component content, especially protein content up to 65 %, and powerful activity.

CN102020705A;CN102020705B describe a method for preparing spirulina protein isolate. The method is characterized by comprising the following steps of: (1) preparing a spirulina cell disrupted, decolorized, deodorized and degreased material; (2) extracting protein by using dilute solution of salt and centrifuging; (3) adjusting the pH untilthe solution is acidic in a gradient and sectional way, performing acid precipitation to obtain the protein, and adjusting the pH until the solution is neutral; and (4) performing ultra-high temperature short-time sterilization and spray drying. The spirulina protein isolate produced by the method has the advantages of light color, light fishy odor, high protein content, high dissolubility and easiness of digestion, and can be used as a high-quality full-value protein nutritional supplement or additive.

CN103519189A;CN103519189B describe a method for preparing food nutrition fortifier by utilizing spirulina.The invention provides a method for preparing food nutrition fortifier by utilizing spirulina. The method comprises the steps of: pretreating spirulina powder by taking ethanol-containing alkaline electrolysis solution as a solvent, wherein the spirulina to solution is 1:10-20; then performing ultrasonic treatment and centrifugation on a sample to obtain supernate and precipitate which are respectively used as food nutrition fortifiers. The preparation basically gets rid of the fishy smell of spirulina, has great change in color, and can be added into food, thereby greatly expanding the application range of the spirulina, breaking through the limit that the spirulina can only be used as tablet and capsule health-care products, and effectively supplementing the nutritional values of fermented and deep-fried food and fruit and vegetable juice.

CN103772482B The present invention uses spirulina (rich in selenium) as the raw material, after being crushed and extracted by the overflow circulation ultrasonic wave, the filter cloth is filtered, and the filtrate is centrifuged at a high speed of 6000 to 18000 rpm at 0 to 40° C. for 10 to 60 minutes, and the supernatant is taken through ultrafiltration The membrane separation system intercepts the required active protein solution, and finally the protein solution is dried by microwave vacuum to obtain the production process and preparation method of spirulina(selenium-enriched) nutritional protein powder. The process and preparation method are simple, easy to operate, low energy consumption, and can be used for industrial production. The obtained product has natural activity, good color, fragrance and nutrient content

CN104479010A describes a process of wall broken spirulina protein powder. The invention discloses a preparation process of wall broken spirulina protein powder. The preparation process of the wall broken spirulina protein powder comprises the following steps of: pulverizing wall broken spirulina with a pulverizer, soaking wall broken spirulina powder in water for 5-10 hours, taking out the soaked wall broken spirulina powder, and pulverizing under ultrasonic waves; soaking spirulina cells with distilled water or a low-salt solution directly so that the spirulina cells swell and break to release wall broken spirulina protein, wherein the wall broken spirulina protein is soaked in distilled water for 10 days, or the wall broken spirulina protein is soaked in the low-salt solution for 3-4 days; weighing 5.99 g of wall broken spirulina protein powder, and soaking in distilled water for 12 days for swelling fully; filtering with a piece of filtering cloth of 74 mu m, centrifuging a filtrate at the temperature of 4 DEG C and 5500 g for 20 minutes, removing spirulina residues, and taking a purplish red supernatant which is the crude extract of wall broken spirulina protein. The preparation process of the wall broken spirulina protein powder is simple, and the prepared wall broken spirulina protein powder has a high nutritional value.

CN104432074B discloses a spirulina powder and a preparation method thereof, which comprises the following steps: adding water to pulverize the spirulina raw material, breaking the wall, preparing a fermentation substrate, fermenting with lactic acid bacteria, freeze-drying and pulverizing after fermentation. Compared with the spirulina powder produced in the prior art, the spirulina powder prepared by the method of the present invention has a 5-9 times higher polypeptide content; it is rich in probiotics, and the number of probiotics > 2×108 cfu/g, while the common spirulina The powder basically does not contain probiotics; compared with ordinary spirulina powder, the content of vitamin B1 is increased by 30%, the content of vitamin B2 is increased by 22%,and the content of vitamin C is increased by 35%. Animal experiments show that the spirulina powder prepared by the method of the present invention has better immune function enhancement than the existing commercially available spirulina powder.

CN104644694A describes a method of ultra micro spirulina powder as well as preparation and application.The invention discloses a preparation process of ultramicro spirulina powder. The preparation process of the ultramicro spirulina powder comprises the following two steps: (1) directly superfine grinding the spirulina; and (2) uniformly mixing the spirulina water extract and the extracted spirulina residue product to obtain the spirulina powder. The average grain size of the spirulina powder is 200-800 nm. The prescription and the preparation process of the tablet, the capsule and the soft capsule of the ultramicro spirulina powder and the common spirulina powder (less than 100 meshes) are created and researched based on the ultramicro spirulina powder. The additive amount of the auxiliary materials is greatly reduced; by adopting the new preparation molding process, the problems of unreasonable prescription of the current spirulina product, many auxiliary materials, multiple varieties, low purity, tedious production process and low product quality can be solved; and the industrial production speed can be quickened and the production efficiency can be increased.

CN106360333 describes a Noodle containing spirulina protein and processing method thereof. The invention provides a noodle containing spirulina protein and a processing method thereof. The noodle is prepared by refining the following raw materials in parts by weight: 0.1-5 parts of spirulina protein, 80-y of wheat flour, 10-20 parts of wheat starch, 0.5-5 parts of table salt, 0.1-0.8 part of sodium carbonate, 0.1-1 part of composite phosphate, 0.1-3 parts of konjac gum, 0.1-1 part of sodium carboxymethyl cellulose, 0.1-1 part of sodium alginate and 20-100 parts of drinking water. The noodle provided by the invention contains the spirulina protein, and the spirulina protein, of which the amino acid composition is very orderly and reasonable, is rich in amino acids essential for a human body; hydrophilic gel prepared by adding the konjac gum, the carboxymethyl cellulose sodium and the sodium alginate greatly improves the mouth feeling of the noodle; the noodle is food which is rich in nutrition and delicious and is very suitable for being used as daily staple food

CN108294299 discloses spirulina powder and a preparation method thereof. The preparation method comprises the following steps of preparing spirulina strain liquid, breeding spirulina, collecting and flushing algae mud, performing freeze drying, and performing crushing. Compared with the spirulina powder prepared by a conventional technique, for the spirulina powder prepared by the method, the protein content achieves 80.5-84.0%, and the beta-carotene content achieves 300-325 mg/100 g;and the spirulina powder is rich in calcium, magnesium and iron elements, the calcium content achieves 400-430 mg/100 g, the magnesium content achieves 330-350 mg/100 g, and the iron content achieves 85-100 mg/100 g. The spirulina powder is applied to the field of health-care foods, and generates good effect of improving immunity for crowds lacking of elements of calcium, iron and magnesium and having low immunity.

CN103519189A describes a method for preparing food nutrition fortifier by utilizing spirulina. The method comprises the steps of: pretreating spirulina powder by taking ethanol-containing alkaline electrolysis solution as a solvent, wherein the spirulina to solution is 1:10-20; then performing ultrasonic treatment and centrifugation on a sample to obtain supernate and precipitate which are respectively used as food nutrition fortifiers. The preparation basically gets rid of the fishy smell of spirulina, has great change in color, and can be added into food, thereby greatly expanding the application range of the spirulina, breaking through the limit that the spirulina can only be used as tablet and capsule health-care products, and effectively supplementing the nutritional values of fermented and deep-fried food and fruit and vegetable juice..

CN1 02020705A describes a method for preparing spirulina protein isolate. The method is characterized by comprising the following steps of: (1) preparing a spirulina cell disrupted, decolorized, deodorized and degreased material; (2) extracting protein by using dilute solution of salt and centrifuging; (3) adjusting the pH until the solution is acidic in a gradient and sectional way, performing acid precipitation to obtain the protein, and adjusting the pH until the solution is neutral; and (4) performing ultra-high temperature short-time sterilization and spray drying. The spirulina protein isolate produced by the method has the advantages of light color, light fishy odor, high protein content, high dissolubility and easiness of digestion, and can be used as a high-quality full-value protein nutritional supplement or additive.

CN101366404A describes a method for preparing spirulina flour. The method comprises the following steps: flour directly passes through a flat sieve to sieve out excellent flour to enter a packing auger; the packing auger stirs the flour uniformly; spirulina powder is filled into a feeder and the flat sieve; the sieved excellent flour simultaneously enters two packing augers to ensure that the flour and the sprirulina powder are stirred more uniformly; the mixed flour is brought into a container by a hoisting machine, and is packaged into a finished product by an electronic quantitative packing scale. With the method, the spirulina powder and the flour are combined to be easily digested by a human body, so that physiological functions of the human body can be balanced to promote metabolism and improve immunity; moreover, the spirulina powder and the flour can be stirred more uniformly by the two packing augers. The spirulina flour is rich in comprehensive nutrition, such as protein, chlorophyll, vitamin, mineral, microelement and the like.

CN1344514A describes a spray drying process. The present invention provides one vacuum freezing and sublimating drying process so as to maintain the activity and effective components of spirulina. The spirulina powder as one kind of health product has high nutrient component content, especially protein content up to 65%, and powerful activity.

Most spirulina protein isolates known in the art occur as dark brown colored powder mass, with strong fishy odor and bitter taste, and thus not suitable for use in the food industry. The product does not have desired properties such as sponginess, porosity, texture, odor and taste. Efforts to use such a spirulina protein product in making alternate meat or milk have been unsuccessful. In terms of protein, only soy milk is comparable to dairy milk. Its protein levels are comparable with dairy milk, with 3.4 g per 100 ml for soy versus 3.5 g per 100 ml of cow’s milk, on average. Other plant-based milks like almond, rice, and coconut contain much less protein compared to soy, whereas relative newcomers hazelnut, hemp and oat milk tend to sit somewhere in between. Plant milks are also sometimes foritifed with vitamins and minerals like calcium, vitamin B12 and vitamin D. Bioavailability is different for fortified nutrients in different beverages. Not all protein in food is created equal. A number known as the digestible indispensable amino acid score (DIAAS) indicates the quality of a protein based on its amino acids ― the building blocks of proteins ― and how well humans digest them. Cow’s milk has a higher protein quality score than soy, but only slightly. The protein quality in other plant-based milks tends to be lower than either dairy or soy milk. To be considered “complete,” a protein must contain all nine essential amino acids, which provides the full mix of essential amino acids our body needs. From muscles to hair, bones to teeth, the body needs protein to be healthy and strong. Soy protein is deficient in Met-Cyst and Phe-Tyr. Calcium present in most plants is not absorbable due to the presence of anti-nutrient phytic acid.

Finally, terms of carbon emissions, almond, oat, soy, and rice milk are all responsible for around a third or less of the emissions associated with dairy milk, with almonds the lowest at 0.7 kg per litre, followed by oat (0.9 kg), soy (1 kg), then rice (1.2 kg). Dairy milk is responsible for 3.2 kg of emissions per litre of milk.

Dairy milk has several disadvatanges, including lactose intolerance. There has been ever increasing demand for an alternative milk that are free from lactose. While there are large number of plant based milk products commercially available, none of them provide adequate nutrion in terms of proteins, vitamins and minerals as in dairy milk. Soy milk has adequate protein, comparable to dairy milk but is deficient in essential nutrients such as Niacin, Pantothenic acid, Phosphorous, Vitamin D. Though it has a small amount of calcium, it not absorbable due to the presence of phytic acid. In addition, the proteins are incomplete due to nondigestible Met-Cys and Phe-Tyr aminoacids. In addition, there is a high risk of allergy. Pea protein compares to whey protein in regards to its high rate of absorption in the body. It is an incomplete protein, low in Methionine, so in order to reach full effectiveness, it should be paired with another protein, such as rice. None of the plant based protein milks provide all 13 essential nutrients that are present in cow’s milk.

Spirulina, a blue-green algae has 65-75% proteins and is packed with a large number of vitamins and minerals. It is considered to be most nutrient-dense plant source. While proteins present in spirulina are considered to be ‘Complete’, the digestibility is much lower than whey and soybean. Significant proportion of nutrients present spirulina are not absorbed in the body as they are entrapped within the cell wall.

Hence, there is need for a plant based milk from which proteins and other nutrients are readily absorbed. The proteins should be ‘Complete’ with respect to the amino acid composition and should contain all 9 essential amino acids and non-essential amino acids. It should also provide all 13 essential nutrients that are part of the cow’s milk. Most importantly, it should be low in fat.

Dairy milk suffers from several disadvantages including lactose intolerance. Replacing dairy milk with a plant based milk is met with many challenges, such as content of protein, quality of protein, digestibility and bioavailability of proteins, presence of all 13 essential nutrients, plant based allergens, presence of anti-nutrients such as phytic acid etc. Most plant based alternative milks, except soy, lack sufficient proteins. Most plant proteins, including soy are not ‘complete proteins’. They deficient in one or more amino acids. For example, soy proteins are deficient with respect to Met-Cyst and Phe-Tyr amino acids. It also deficient in several essential nutrients. Phytic acid present in soy prevents absorption of calcium.

Spirulina is a blue-green algae, regarded as ‘super food’ has upto 70% proteins. It is considered to be richest source of protein. The proteins in spirulina contain all 9 essential amino acids and 11 non-essential amino acids. Since all these proteins are fully absorbable, spirulina proteins are classified as ‘complete protein’. In addition to proteins, spirulina has most of the essential nutrients including vitamins, minerals, omega-3 fatty acids, GLA and zeaxanthin. Spirulina is considered to be ‘nutrient dense’ super food.

The main drawback of spirulina proteins is that its digestibility is just about 80%, as compared to 95-98% of whey proteins. One possible reason for lower digestibility could be the fact that these proteins are entrapped within a thick cell wall. Another reason for its poor bioavailability is the fact that the proteins are tightly coiled and entangled, such that its digestion in stomach and intestine are generally incomplete. For the same reasons, bioavalability of most other nutrients are adversely affected.

Other drawbacks of spirulina proteins are a. undesirable taste, b. fishy odor and c. dark brown/blue color.

Due to these drawbacks, all previous attempts to use spirulina as an alternative milk were unsuccessful.

### SUMMARY

Provided herein is an alternative milk composition of spirulina which comprises digestible, bioavailable proteins, vitamins and minerals. The proteins, vitamins and minerals present in spirulina biomass are rendered digestible and bioavailable by the unique homogenization process described herein, followed by thermo-chemical treatment. The alternative milk described herein, made with digestible, bioavailable proteins, vitamins and minerals, may be formulated with colorant, sweetener, flavor and gelling agent. The milk so obtained is packed with digestible and bioavailable proteins, vitamins and minerals is free from fishy odor and bitter taste associated with prior art spirulina compositions..

### DETAILED DESCRIPTION

The present inventors have surprisingly found that by treating spirulina whole cells as described herein it is possible to make the proteins fully digestible and bioavailable. The proteins are fully water soluble and are completely devoid of fishy odor and bitter taste. Most importantly, the protein product retains all other important essential nutrients present in the spirulina whole cells. The process described herein comprises the steps of
a. suspending the wet biomass of spirulina in alkaline water;
b. disruption or lysis of the cells;
c. thermal treatment under pressure;
d. centrifugation to separate cellular debris; and
e. spray drying the resultant mother liquor.

The resultant protein powder can be easily formulated into a milk by known methods.

The milk so obtained has properites similar to that dairy milk and its proteins are readily water soluble, ‘complete’ and fully digestible. The milk so produced has no fishy odor or undesirable taste.

The term ‘disruption or lysis’ of the cells defines the process of breaking the cell walls and/or membranes to release intracellular fluids containing molecules or particles of interest, such as proteins.

The term ‘thermal treatment’ of proteins defines configurational changes in the thermodynamically stable native structure of the protein via unfolding or alteration of the quaternary, tertiary, or secondary structure as a response to heat exposure.

The term ‘digestible protein’ defines a protein that is capable of undergoing proteolytic cleavage in the gastrointestinal tract, and subsequent absorption into the body.

The term ‘complete protein’ defines a whole protein is a food source of protein that contains an adequate proportion of each of the nine essential amino acids necessary in the human diet.

In one of the embodiments described herein, the biomass of spirulina is a wet mass obtained after clarification of the harvested algae. In another embodiment, the biomass is spray-dried following harvest.

In yet another embodiment, cell disruption of the spirulina algae is carried out by ultrasonification.

In yet another embodiment, the cell disruption is through enzymes.

In yet another embodiment, the thermal treatment of spirulina is by heating the lysed cells at 56-65° C. for 30-60 mins under atmospheric pressure.

In yet another embodiment, the thermal treatment of spirulina is by heating the lysed biomass at 110-130° C. at 1.1-1.3 kg/sq cm pressure.

In yet another embodiment, the mother liquor after centrifugation is spray dried at 70-120° C. inlet temperature and 50-80° C. outlet temperature.

In yet another embodiment, the milk composition made with spray dried powder of spirulina, contains one or more of a creamer, a colorant, a sweetener, a flavoring, a preservative, and a jelling agent.

While the compositions and methods herein have been described in terms of specific illustrative embodiments, any modifications and equivalents that would be apparent to those skilled in the art are intended to be included within the scope of the methods and compositions herein. The details of the methods and compositions herein, its objects, and advantages are explained hereunder in greater detail in relation to non-limiting exemplary illustrations.

### EXAMPLE

Fresh spirulina biomass is supended in deminerized water and homogenized using high pressure homogenizer. The biomass suspension is subjected to ultrasonication for 30 mins. The temperature rises to 75° C. The suspension is cooled to ambient temperature and the pH of the suspension is adjusted to 10-11.5 with caustic lye and stirred for 30 mins. The biomass suspension is then subjected to thermal treatment at 130° C. at 1.3 kg/sq cm pressure for 20 mins. The suspension is centrifuged to remove cell wall debris and the pH of the mother liquor is adjusted to 7.5-8 with an acidulant and spray dried with an inlet temperature of 90-110° C. and outlet temperature of 70-80° C.

The spray dried powder is formulated in to a milk product by mixing 10 g of powder with purified water to form a 10% solution, mixed with colorant, flavor and sweetener. The milk product so formed has the following nutrients:
Proteins 7.2 g
Fats 0.1 g
Carbohydrates 2.2 g
Calcium 70 mg (7% DV)
Phosphorous 80 mg (19% DV)
Beta Carotene 6.6 mg (55% DV)
Thiamine 0.01 mg (1% DV)
Riboflavin 0.9 mg (35% DV)
Vitamin C 0.025 mg
Niacin 1.6 mg (10% DV)
Pantothenic acid 10ug (1% DV)
Vitamin B12 24ug (51% DV)
Folic acid 5ug (1% DV)
Potassium 140 mg (4.5% DV)
Sodium 960 mg (41% DV)
Selenium 10ug (18% DV)
Zinc 0.1 mg (1% DV)
Iron 4.5 mg (25% DV).

Amino acid profile of the protein:
Phenylalanine 3.9%
Methionine 1.96%
Threonine 4.1%
Valine 4.85%
Isoleucine 4.5%
Leucine 7.2%
Lysine 3.95%
Histidine 1.3%
Tryptophan 1.06%
Alanine 5.75%
Arginine 4.96%
Glutamic Acid 9.73%
Glycine 3.68%
Proline 3.1%
Cysteine 0.82%
Tyrosine 3.62%.

The highly digestible milk so produced was creamy pale yellow in color, free from fishy odor and bitter taste.

1. A process for preparing an alternative milk composition comprising the steps of:
providing spirulina biomass;
homogenizing the spirulina biomass under alkaline conditions and high pressure to form a homogenized biomass.

2. The process of claim 1, wherein the homogenizing step is carried out by ultrasonic treatment of the spirulina biomass at 40-110 kHz frequency at 80-120 W power for 1-5 hours.

3. The process of claim 1, wherein the homogenizing step includes the step of heating the homogenized spirulina biomass at 110-135° C. and 1.1-1.3 kg/sq cm pressure for 20-30 mins.

4. The process of claim 1, wherein the homogenizing step includes the step of treating the spirulina biomass with an alkali to achieve a pH of 10.8-11.5.

5. The process of claim 1, further comprising the step of spray-drying the homogenized biomass.

6. The process of claim 1, further comprising the steps of
precipitation at pH 3.5-4.5,
centrifugation,
drying the resultant solids at 80° C. for 8 hrs.

7. An alternative milk product produced by the process of claim 1.

8. The alternative milk product of claim 7, which further comprises one or more ingredients selected from the group consisting of a colorant, a sweetener, a flavor, and a gelling agent.
